(19) United States
(12) Patent Application Publication
(10) Pub. No.: US 2006/0057088 A1
(43) Pub. Date: Mar. 16, 2006

(54) Vaccine for hair removal

(76) Inventor: Nikolai Tankovich, San Diego, CA (US)

Correspondence Address: JOHN R. ROSS, PO BOX 2138, DEL MAR, CA, 92014, US

(21) Appl. No.: 11/229,088

(22) Filed: Sep. 16, 2005

Related U.S. Application Data
(60) Provisional application No. 60/610,225, filed on Sep. 16, 2004.

(51) Int. Cl. A61K 39/395; A61K 8/64; A61K 39/00; A61K 39/08

(52) U.S. Cl. 424/70.1; 424/130.1; 424/185.1; 424/239.1; 424/70.14

(57) ABSTRACT

A process for delivering hair removal fluids to hair ducts. In a preferred embodiment the fluid is a vaccine for slowing or stopping hair growth and a process for applying the vaccine or other hair growth vaccines to the skin region having unwanted hair and a method of delivering the vaccine to skin regions of unwanted hair. The preferred vaccine consists of an antigen cocktail of weakened or essentially dead elements of hair follicles for stimulating antibody responses. The preferred vaccine is prepared from patient's own hair papilla or bulge area stem cells. In other embodiments vaccines are made from extracts of papilla cells cultivated in a laboratory. The vaccines produce antibodies and these antibodies attack and destroy hair papilla and block bulge area cells that would otherwise produce or stimulate a growth of new papilla cells. Even if any new papilla cells appear, the circulating antibodies of the vaccine-activated immune system attack and destroy them also. Other hair removal fluids that can be applied with the process of this invention are also described.

This Application claims the benefit of Provisional Patent Application, Ser. No. 60/610,225 filed Sep. 16, 2004. The present invention relates to processes for hair removal and especially to such processes utilizing vaccines for hair removal.

### BACKGROUND OF THE INVENTION

### Unwanted Hair

Getting rid of unwanted hair is a very big business in the United States. Most men shave their faces nearly every day. Women and girls shave their legs and under their arms and other places. Some women and some men undergo the pain of having their hair ripped out by the roots using waxing techniques. Lasers are also regularly used for hair removal.

### Vaccines

Use of vaccines is a very common way of building up the immune system to fight infection. The vaccine is given by injection (usually under the skin, orally or topically). Once the immune system becomes aware of the antigens in the vaccine, it responds by making antibodies. Vaccines have been applied to the skin in attempts to prevent or cure skin diseases. These vaccines prevent or slow down growth of cancer cells.

What is needed in a vaccine for preventing growth and replication of hair cells.

### SUMMARY OF THE INVENTION

The present invention provides a process for delivering hair removal fluids to hair ducts. In a preferred embodiment the fluid is a vaccine for slowing or stopping hair growth and a process for applying the vaccine or other hair growth vaccines to the skin region having unwanted hair and a method of delivering the vaccine to skin regions of unwanted hair. The preferred vaccine consists of an antigen cocktail of weakened or essentially dead elements of hair follicles for stimulating antibody responses. The preferred vaccine is prepared from patient's own or allogeneic hair papilla or bulge area stem cells. In other embodiments vaccines are made from extracts of papilla cells cultivated in a laboratory. The vaccines produce antibodies and these antibodies attack and destroy hair papilla and block bulge area cells that would otherwise produce or stimulate a growth of new papilla cells. Even if any new papilla cells appear, the circulating antibodies of the vaccine-activated immune system attack and destroy them also. Other hair removal fluids that can be applied with the process of this invention are also described.

### BRIEF DESCRIPTION OF THE DRAWINGS

FIG. 1 is a cross-sectional representation of human skin.

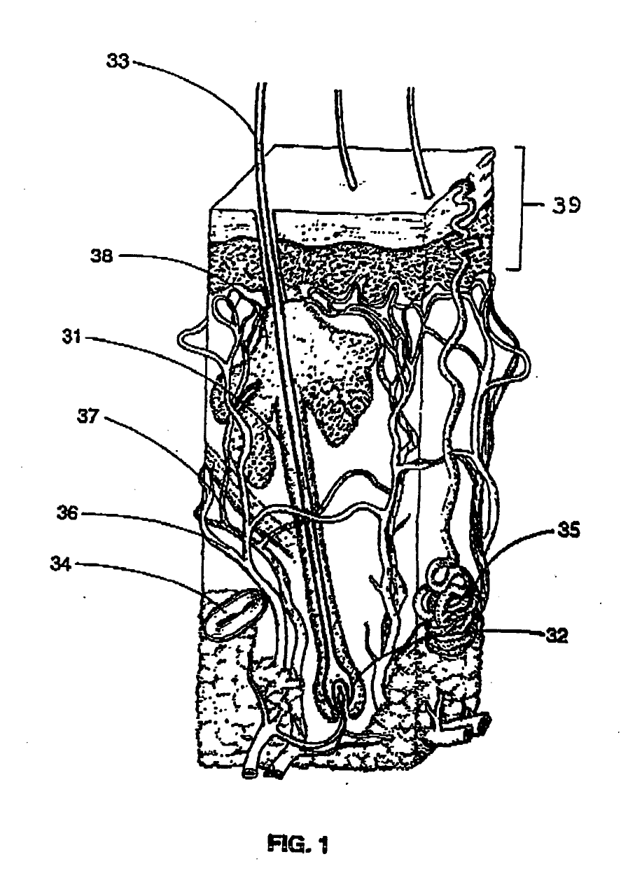

FIGS. 2A-B shows a process of hair waxing in the liquid medium containing vaccine to be delivered.

FIG. 3 shows a process of pulling hair in the medium with vaccine.

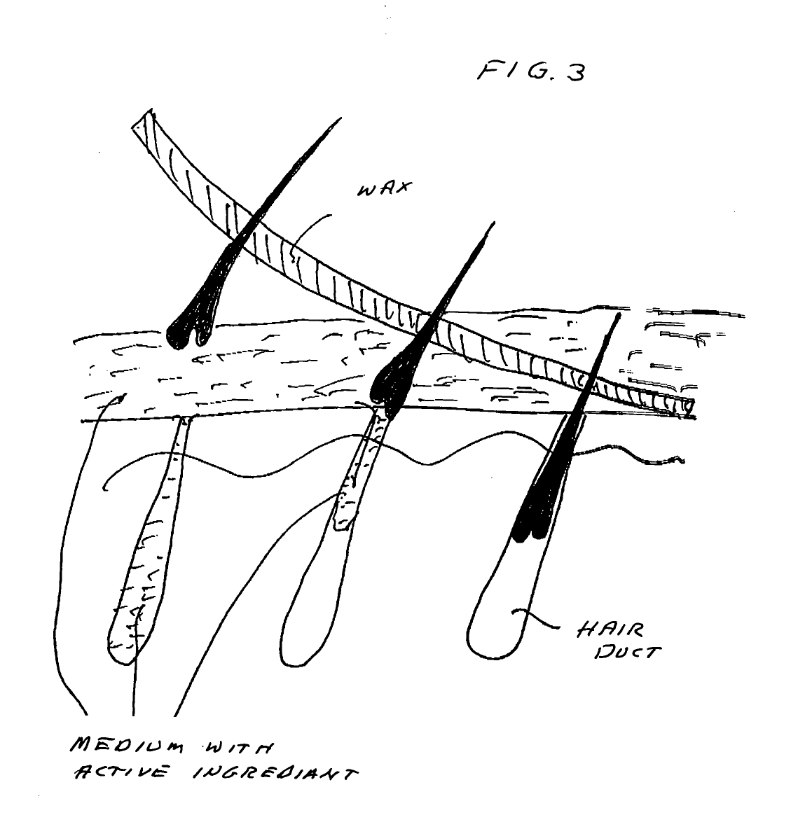

FIG. 4 shows a device for hair pulling with canister containing vaccine or encapsulated vaccine, melting from the body temperature membrane, covering cup with separating membrane.

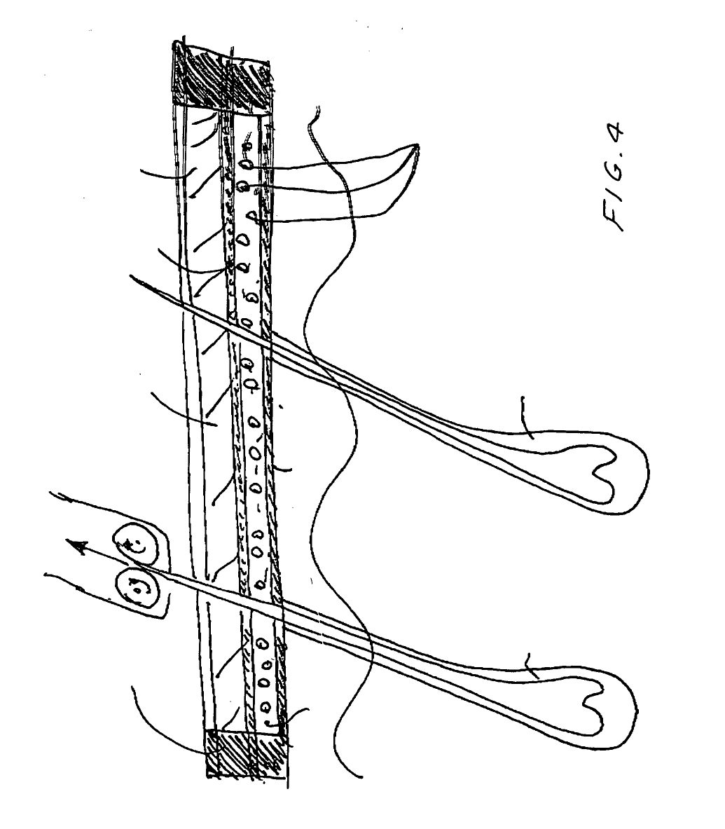

### DETAILED DESCRIPTION OF PREFERRED EMBODIMENTS

Preferred embodiments of the present invention can be described by reference to the drawings.

### Vaccine Preparation

A preferred vaccine for slowing or stopping hair growth is prepared as follows:
1. One tube of Digestion Solution (0.25% Collagenase in Hank's Balanced Salt Solution) per estimated 1 cm³ of specimen to be processed is placed in 37° C. water bath.
2. The sample transport medium is removed from the container by a 20 ml pipet.
3. Approximately 10 ml of sterile purified buffer solution (PBS) is poured into container by a 10 ml pipet.
4. The specimen is washed by pipeting with PBS in and out 6-10 times.
5. The PBS is removed from container by a 10 ml pipet.
6. Steps 5-7 is repeated 6-10 times for a total of six or more as needed.
7. One tube (20 ml) of Digestion Solution per estimated 1 cm³ of specimen to be processed is prepared.
8. The specimen is transferred or scraped into the 50 ml tube with Digestion Solution by scalpel.
9. The magnetic stir bar is placed into the tube with Digestion Solution by sterile forceps.
10. The tube with Digestion Solution is closed tightly, sealed with parafilm and placed on the magnetic stand in the 37° C. water bath.
11. The magnetic stirrer is turned on.
12. The magnetic stirring is activated by slowly turning control knob clockwise until whirling movement of liquid in the tube becomes visible but not so fast as to produce aeration or splashing.
13. The magnetic stirring is maintained for 2 hours, or until all tissue fragments are broken, whichever comes first.
14. The magnetic stirring is stopped.
15. The 50 ml tube is removed from magnetic stand, wiped with cover sponge and placed under a sterile hood.
16. The digested specimen solution is filtered through a double layer of sterile Nitex screen placed into a sterile funnel, and collected into a sterile 50 ml centrifuge tube.
17. The tube with the filtered specimen solution is centrifuged at 1500 rpm for 5 minutes at 5° C.
18. The supernatant is decanted, the remaining pellet is re-suspended with Hank's Balanced Salt Solution (10 ml per 1 cm³ of digested tissue) and mixed thoroughly.
19. About 0.1 ml of the cell suspension is used for cell count.
20. The culture tubes are centrifuged at 1500 rpm for 5 minutes at 5° C.
21. The supernatant is decanted and the pellet is re-suspended with the amount of Culture Medium to adjust the cell concentration to 1×10⁶/ml and mixed thoroughly.
22. The cell suspension is transferred into the tissue culture flasks. The size of the flasks is selected depending on the total volume of culture medium to be used.
23. The flasks are placed into 5% CO2 incubator.

### Preferred Process for Delivering the Vaccine

A preferred process for the delivery of vaccine into the skin region of unwanted skin is described below.

The important step in this embodiment is to physically remove the hair shafts from the hair ducts in the skin section to be treated with the skin surface covered with vaccine to be delivered in the liquid medium. This is accomplished using a well-known temporary hair removal procedure known as waxing. I prefer using a commercially available wax marketed by Slect Spa Source of Sausilito, Calif. under the trade name Nature's Own Pine Wax although a wide variety of such waxes are available and would be satisfactory.

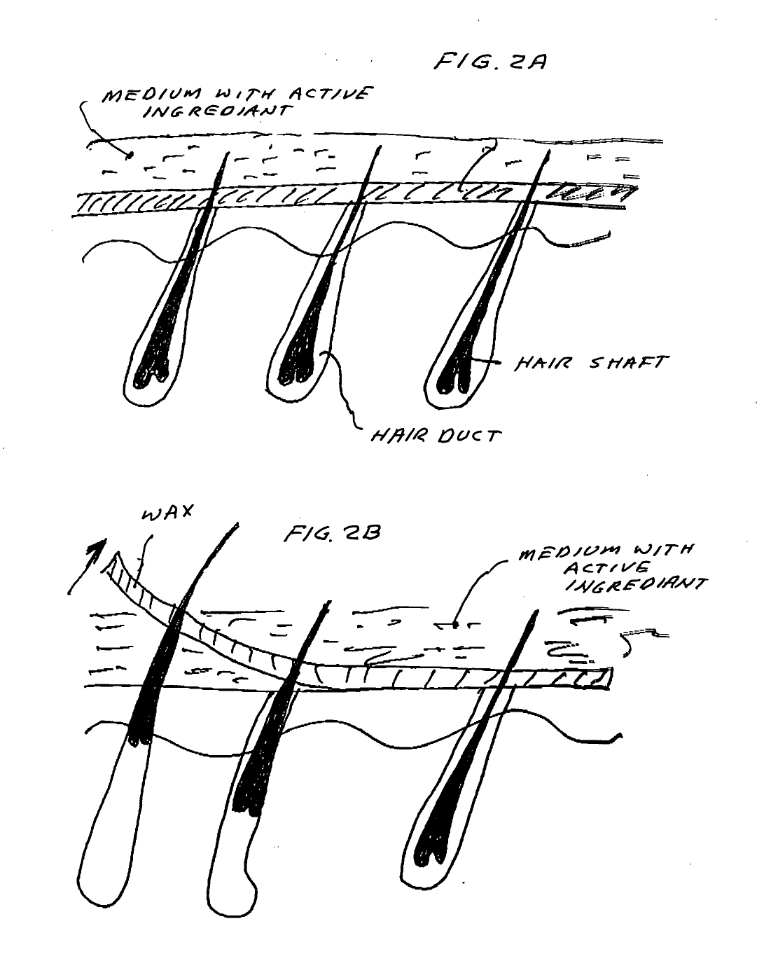

The first step of the procedure is to wash a section of the skin to be treated with methyl alcohol and allowed to dry. A section of skin with growing hairs is depicted in FIG. 2A. Next step is to apply a liquid wax to the surface of the skin with a spatula, cover with a waxing paper stripe. The wax is allowed to dry and the paper and hair on the skin are allowed to adhere to the wax. The skin is immersed into a medium containing vaccine and the paper stripe is pulled up carrying the wax and hairs.

When hairs are in the process of pulling out from the hair ducts the negative pressure is created inside the duct. At the moment when hair bulb is leaving a hair duct, the surrounding fluid containing vaccine rushes into empty hair canals filling it from the top to the bottom as shown in FIGS. 2B and 2C.

### Other Vaccines

Other ther vaccines can be injected into the skin by utilizing other fluids as the surrounding fluid. For example the surrounding fluid could include anti-proliferation agents, antibodies, hair growth factors, allogeneic and autologous vaccines against hair follicle cells, proteins, specific and non-specific receptors, blood vessel material, melanosomes, anti-cancer, anti-inflammatory, cardiovascular, anesthetic, anti-asthma and allergy, diabetic drugs, hormones, oligonucleotidies and other antisence compositions, cells and its fragments, microdevices, depot of wanted material, botox, collagen or collagenase and dyes.

### Microcarriers

In another embodiment of the invention, the chemical is entrapped or encapsulated within microcarrier particles in a formulation suitable for topical administration, such as a formulation of liposomes or coacervate microcapsules. As shown in FIG. 2, the microcarrier particles containing the chemical are applied topically to the skin surface and induced to penetrate into a hair duct during waxing or other hair pulling out technique or device.

The lining of hair ducts does not have an epithelial barrier layer, such as the stratum corneum. Consequently, the lipids in lipid-based particles, such as liposomes, enhance penetration of the prodrug across the cell walls of papilla cells, stem cells, and keratinocytes in the infiltrated hair ducts. Then, irradiation of the skin surface with sunlight as described above activates the photochemical prodrug so as to damage these hair growth cells.

To accommodate this function, the microcarrier particles are sized large enough to avoid absorption across the stratum corneum at the skin surface, but small enough for entry and passage into the hair duct. For application to humans, the diameter of the microcarrier particles is generally less than about 70 μm, for example about 10 μm to about 50 μm because the diameter of hair ducts in humans is in the size range from about 70 μm to about 1 micron. Methods are well known for encapsulating an active agent within a microcarrier particle, such as a liposome or a microcapsule. For example, there are at least three types of liposomes. Multivesicular liposomes (MVL) are man-made, microscopic lipid vesicles comprising lipid membranes enclosing multiple non-concentric aqueous chambers. Multilamellar liposomes or vesicles (MLV) have multiple “onion-skin” concentric membranes, in between which are shell-like concentric aqueous compartments. Multilamellar liposomes and multivesicular liposomes characteristically have length-weighted mean diameters in the micrometer range, usually from 0.5 to 25 μm. Unilamellar liposomes or vesicles (ULV) are liposomal structures having a single aqueous chamber, usually with a mean diameter range from about 20 to 500 nm.

Multilamellar and unilamellar liposomes can be made by several relatively simple methods. The prior art describes a number of techniques for producing ULV and MLV (for example U.S. Pat. No. 4,522,803 to Lenk; U.S. Pat. No. 4,310,506 to Baldeschweiler; U.S. Pat. No. 4,235,871 to Papahadjopoulos; U.S. Pat. No. 4,224,179 to Schneider; U.S. Pat. No. 4,078,052 to Papahadjopoulos; U.S. Pat. No. 4,394,372 to Taylor; U.S. Pat. No. 4,308,166 to Marchetti; U.S. Pat. No. 4,485,054 to Mezei; and U.S. Pat. No. 4,508,703 to Redziniak). Methods for making multivesicular liposomes are disclosed in Kim et al., Biochem. Biophys. Acta, 728:339-348, 1983). For a comprehensive review of various methods of ULV and MLV preparation, refer to Szoka, et al. Ann. Rev. Biophys. Bioeng. 9:465-508, 1980. Also contemplated within the scope of this invention is a composition for topical application to a skin surface for inhibiting hair growth on the skin surface. The composition comprises a sunlight-activated prodrug encapsulated in a microcarrier particle, such as a liposome or microsphere. The diameter of the particles is generally less than 70 μm, for example about 10 μm to about 50 μm. In one embodiment, the sunlight activated prodrug is selected from the group consisting of photophrine II, amino levulenic acid, and tetracycline. The composition further comprises a physiologically acceptable carrier suitable for topical application. The carrier may comprise any conventional topical formulation base, such as those described in Remington's “Pharmaceutical Sciences,” 17^{th} Edition (Mack Publishing Co., Pa.), the disclosure of which is incorporated by reference. A lotion, suspension in oil, solution, cream, ointment, gel, aerosol, or nebulized formulation are representative of the topical compositions of this invention.

This method for inhibiting the growth of unwanted hair provides the advantage of home treatment because the light source used to activate the photochemical prodrug is sunlight. A topical composition containing the sunlight-activated prodrug can be self-administered, and, after a rest period to allow accumulation of the prodrug in hair-growth cells lining hair ducts, a simple sun bath is all that is required to activate the prodrug and thereby inflict sufficient damage to inhibit hair growth on a long term basis.

### A Method for Inhibiting Hair Growth Using Anti-Proliferative Agents

It is known to infiltrate photoactivated chemical compounds, such as porphyrin and chlorin derivatives, into hair ducts, and then to illuminate them with light at a wavelength that causes the photochemicals to release species harmful to cells in hair follicles responsible for hair growth. However, the light sources used for illumination, such as lasers, are expensive and usually require trained operators to avoid unwanted damage to skin and eyes.

This method provides a method for inhibiting growth of unwanted hair on a section of skin by topically applying an anti-proliferative agent to the section of skin to be treated. The anti-proliferative agents do not require activation by any type of light source. Normal hair growth will recommence once the treatment is withdrawn. During application of the anti-proliferative agent, care is taken to assure that at least a portion of the anti-proliferative agent is delivered into hair ducts on the section of skin to be treated to inhibit hair growth. Preferably the anti-proliferative agent is delivered along the full length of the hair duct, which length varies depending upon the bodily location of the skin section to be treated (i.e., on the face, legs, or arms). The depth of the hair duct also varies for an individual hair depending upon the phase of the hair growth cycle in which it is found. During the mature anagen phase, for example anagen V and VI, the hair follicle is fully extended (to a depth of 3.0 to about 5.0 mm), and the distance to the bottom of the follicle from the skin surface is about twice that during the telogen phase of the hair growth cycle. Therefore it is advantageous to synchronize the growth cycle of the hairs to be treated before application of the anti-proliferative agent to the area to be treated for inhibition of hair growth. Methods for synchronizing the hair growth cycle are disclosed above in Section No. 1 herein.

The anti-proliferative agent is applied to the skin surface in any suitable topical formulation, such as a lotion, cream or gel. Suitable formulations preferably are designed to aid in delivery of the anti-proliferative agent into hair ducts, and may therefore, include one or more chemical agents that will reduce surface tension, such as a surfactant.

Anti-proliferative agents useful in the practice of this invention include small molecules as well as macromolecules, such as proteins or enzymes, that interfere with or interrupt in any way the cycle of cell proliferation. Representative examples of anti-proliferative agents useful in the compositions and methods of the present invention include methotrexate, doxorubicin, taxol, tumor necrosis factor, chlorambucil, interleukins, etoposide, cytarabine, fluorouracil, vinblastine. The mechanism of action of the anti-proliferative agent is immaterial other than that it interferes with or interrupts the cycle of cell proliferation. For example, methotrexate, aminopterin and cytosine arabinoside (also known as cytarabine and Ara-C) are cell cycle-specific antimetabolites that kill cells only when they are synthesizing DNA. Fluorouracil inhibits formation of both DNA and RNA. Methioninease is an enzyme that inhibits uptake of methionine by hair papilla cells proliferating at a high rate.

Hair ducts are not lined by an epithelial barrier layer, such as the stratum corneum, but do contain rapidly proliferating hair papilla cells, stem cells, keratinocytes, and endothelial blood vessels, which generate hair growth. These cells in the hair duct responsible for hair growth are the fastest growing cells in the body, aside from tumor cells. Due to the absence of a barrier layer in the hair duct, the anti-proliferative agents are preferentially take up by these hair growth cells, which have a high metabolic rate. Entry of the anti-proliferative agents into other, more slowly growing cells, is at a substantially slower rate. Consequently, the anti-proliferative agents are preferentially absorbed into the target hair growth cells, with the result that hair growth is inhibited.

Application of the anti-proliferative agent to the skin surface is repeated at spaced intervals of hours or days until hair growth is inhibited. Generally, the anti-proliferative agent is applied two times daily for so long as it is desired to inhibit the growth of hairs from the treated portion of the skin. Once treatment is stopped, hairs in the treated section of skin will commence a normal growth pattern.

In one embodiment, the anti-proliferative agents are encapsulated in lipid-based particles, such as liposomes or microcapsules, for application to the skin surface. The lipid-based particles are sized small enough to enter into hair ducts in the section of skin, but large enough not to be absorbed across the stratum corneum. Care is taken during application of the lipid-based particles to assure that at least a portion of the drug-bearing particles enters into the hair ducts. For example, the micro particles can be formulated in a physiologically acceptable carrier containing one or more chemical agents that will aid entry of the particles into hair ducts. It is believed that administration of the anti-proliferative agents encapsulated in lipid-based particles will increase uptake of the anti-proliferative agent. As the lipid-based particles begin to break down in the hair duct, both the encapsulated drug and lipids from the bilayers of the lipid-based particles are released. These lipid byproducts can aid in the delivery of the released drug across the membranes of the target cells. Methods for obtaining an active agent encapsulated in lipid particles, such as liposomes and microcapsules are well known in the art and are referred to above in Section No. 8 herein.

Lipid-based particles, such as liposomes, deliver the encapsulated agent slowly within the hair duct, so that the cells lining the hair duct are bathed in the anti-proliferative agent over an extended period of time, generally over a period of hours or even days. Slow release of the anti-proliferative agent from the lipid-based particles is particularly advantageous for those agents that interfere with a particular step in the proliferation cycle of the cells, such as formation of DNA and/or RNA, because not all cells enter mitosis at the same time.

The dose of the anti-proliferative agent administered, whether encapsulated or unencapsulated, can vary from about a few picomoles to about several hundred millimoles. The desirable dose of anti-proliferative agent per unit area of skin treated is a hair growth-inhibiting amount, and will vary depending upon such characteristics as the stage of target hairs in the hair growth cycle at the time of administration, the age and condition of the subject, the particular properties of the agent, and the dosage schedule. In general, the dosage range of the anti-proliferative agent appropriate for topical application to humans is in the range of about 0.001 to about 6,000 mg/m² of body surface area, generally applied in a cream, ointment or solution containing about 10% of the anti-proliferative agent by weight. While doses outside the foregoing dose range may be given, this range encompasses the breadth of use for most anti-proliferative agents useful for inhibiting hair growth. The dose range for a particular anti-proliferative agent can be easily ascertained as previously described.

The present invention provides the advantage over other hair removal procedures that no specialized equipment is required to inhibit growth of unwanted hair. No lasers, razors, depilatory needles, etc., are required for safe and temporary inhibition of hair growth. The anti-proliferative agent is repeatedly applied onto the surface of skin so as to cause the anti-proliferative agent to enter hair ducts therein, and hair growth will recommence upon cessation of the treatment. The embodiment of the invention in which the anti-proliferative agent is administered in a slow release lipid-based formulation provides convenience by reducing the number of times the formulation must be used to accomplish the goal of inhibiting hair growth.

While the present invention has been described in terms of preferred embodiments persons skilled in the art of hair removal will recognize that many changes and modifications could be made without departing from the basic concepts of the invention. For example, it is possible that vaccines may stimulate patient's T lymphocytes against papilla antigens. In that case the T cells will destroy hair papilla by direct killing or via production of cytokines, which can activate the cytotoxic cells. A successful vaccine immunization may create a pool of immunological memory cells. Those cells may able to maintain immunity to hair papilla over considerable periods of time. If necessary or desired, the patient may receive repeated cycles of immunization to replenish the existing memory cell pool. The frequency of vaccine treatments can be determined based on the immunomonitoring studies, which will evaluate a potency of patient's immune system following the vaccine treatment. Vaccine treatment regimen is usually well tolerated by most of the patients; however, the individual results may vary and all cases of toxicity and adverse effects will be carefully documented and reported to the FDA. Prior to treatment the patients will be requested to sign a consent form explaining all the risks involved. The benefits of the hair papilla vaccine will be evaluated in the context of other available treatment options.

What is claimed is:

1. A process for delivering to hair ducts of skin region with unwanted hair a hair removal fluid for slowing or stopping hair growth comprising the steps of:
A) emersing the skin region in the hair removal fluid,
B) pulling out hair from the skin region and
C) allowing the hair removal fluid to flow into the portion of the skin vacated by the hair.

2. The process of claim 1 wherein said hair removal fluid comprises a vaccine for slowing or stopping hair growth.

3. The process of claim 2 wherein the vaccine comprises an antigen cocktail of weakened or essentially dead elements of hair follicles for stimulating antibody responses.

4. The process of claim 3 wherein said preferred vaccine is prepared from patient's own hair papilla or bulge area stem cells.

5. The process of claim 2 wherein said vaccines are made from extracts of papilla cells cultivated in a laboratory.

6. The process of claim 1 wherein said vaccines produce antibodies and these antibodies attack and destroy hair papilla and block bulge area cells that would otherwise produce or stimulate a growth of new papilla cells. Other hair removal fluids that can be applied with the process of this invention are also described.

7. The process of claim 1 wherein said hair removal fluid is a hair removal fluid chosen from the following group of fluids: anti-proliferation agents, antibodies, hair growth factors, allogeneic and autologous vaccines against hair follicle cells, proteins, specific and non-specific receptors, blood vessel material, melanosomes, anti-cancer, anti-inflammatory, cardiovascular, anesthetic, anti-asthma and allergy, diabetic drugs, hormones, oligonucleotidies and other antisence compositions, cells and its fragments, microdevices, depot of wanted material, botox, collagen or collagenase and dyes.

8. The process of claim 1 wherein said hair removal fluid comprises an anti-proliferative agent.

9. The process of claim 1 wherein said hair removal fluid comprises a hair discoloration vaccine.

10. The process of claim 1 wherein said hair removal fluid comprises a hair color restoration vaccine.
